SEQUENCE LISTING (39 sequences)

SEQ ID NO: 1 - DNA, 22 bp, Artificial sequence
  Description of the artificial sequencePrimer
gaccgaaacaatgtggaattgg

SEQ ID NO: 2 - DNA, 23 bp, Artificial sequence
  Description of the artificial sequencePrimer
gccatcttcatgttggagctttt

SEQ ID NO: 3 - DNA, 20 bp, Artificial sequence
  Description of the artificial sequencePrimer
tgtggaacgggaaaaggaca

SEQ ID NO: 4 - DNA, 20 bp, Artificial sequence
  Description of the artificial sequencePrimer
cccgatatcctccctgatcg

SEQ ID NO: 5 - DNA, 20 bp, Artificial sequence
  Description of the artificial sequencePrimer
gcgttaatggcattcgacca

SEQ ID NO: 6 - DNA, 20 bp, Artificial sequence
  Description of the artificial sequencePrimer
tttcgccattcgcattgtct

SEQ ID NO: 7 - DNA, 24 bp, Artificial sequence
  Description of the artificial sequencePrimer
tttcaccatgaaggggtagatgtt

SEQ ID NO: 8 - DNA, 25 bp, Artificial sequence
  Description of the artificial sequencePrimer
tttcccttttctacgcactaaatgt

SEQ ID NO: 9 - DNA, 20 bp, Artificial sequence
  Description of the artificial sequencePrimer
tccagaaccttgaccgaacg

SEQ ID NO: 10 - DNA, 20 bp, Artificial sequence
  Description of the artificial sequencePrimer
gtcattgcgctgccattctc

SEQ ID NO: 11 - DNA, 20 bp, Artificial sequence
  Description of the artificial sequencePrimer
agtctccccgccatattctc

SEQ ID NO: 12 - DNA, 20 bp, Artificial sequence
  Description of the artificial sequencePrimer
gccgacaatcaaatcatcct

SEQ ID NO: 13 - DNA, 21 bp, Artificial sequence
  Description of the artificial sequencePrimer
tgaaatcggctcaggaaaagg

SEQ ID NO: 14 - DNA, 23 bp, Artificial sequence
  Description of the artificial sequencePrimer
cgtcaattcctgcatgttttaag

SEQ ID NO: 15 - DNA, 20 bp, Artificial sequence
  Description of the artificial sequencePrimer
gtgtcgcccttattcccttt

SEQ ID NO: 16 - DNA, 20 bp, Artificial sequence
  Description of the artificial sequencePrimer
ataataccgcgccacatagc

SEQ ID NO: 17 - DNA, 25 bp, Artificial sequence
  Description of the artificial sequencePrimer
gcctttttaaagaccgtaaagaaaa

SEQ ID NO: 18 - DNA, 25 bp, Artificial sequence
  Description of the artificial sequencePrimer
ccacatcttgcgaatatatgtgtag

SEQ ID NO: 19 - DNA, 20 bp, Artificial sequence
  Description of the artificial sequencePrimer
gtcactatggcgtgctgcta

SEQ ID NO: 20 - DNA, 20 bp, Artificial sequence
  Description of the artificial sequencePrimer
ggtgatgtcggcgatatagg

SEQ ID NO: 21 - DNA, 24 bp, Artificial sequence
  Description of the artificial sequencePrimer
acagagatattttaggggctgaca

SEQ ID NO: 22 - DNA, 26 bp, Artificial sequence
  Description of the artificial sequencePrimer
cttctaaacttcctgtatgcaattct

SEQ ID NO: 23 - DNA, 22 bp, Artificial sequence
  Description of the artificial sequencePrimer
agagccttgggaagatgaagtt

SEQ ID NO: 24 - DNA, 24 bp, Artificial sequence
  Description of the artificial sequencePrimer
gccacactatcataaccactaccg

SEQ ID NO: 25 - DNA, 20 bp, Artificial sequence
  Description of the artificial sequenceOligonucleotide
ggcatcgttccaaagaatgt

SEQ ID NO: 26 - DNA, 20 bp, Artificial sequence
  Description of the artificial sequenceOligonucleotide
gaaagctgcctgttccaaag

SEQ ID NO: 27 - DNA, 20 bp, Artificial sequence
  Description of the artificial sequenceOligonucleotide
gaagcaaatggatggttcgt

SEQ ID NO: 28 - DNA, 20 bp, Artificial sequence
  Description of the artificial sequenceOligonucleotide
tcgaaaactgggaagtcgaa

SEQ ID NO: 29 - DNA, 20 bp, Artificial sequence
  Description of the artificial sequenceOligonucleotide
attgttgtgcacgacgacat

SEQ ID NO: 30 - DNA, 20 bp, Artificial sequence
  Description of the artificial sequenceOligonucleotide
ggtgcgatacagggtctgtt

SEQ ID NO: 31 - DNA, 20 bp, Artificial sequence
  Description of the artificial sequenceOligonucleotide
caaaacgctcattggcatta

SEQ ID NO: 32 - DNA, 20 bp, Artificial sequence
  Description of the artificial sequenceOligonucleotide
tttgccttcctgtttttgct

SEQ ID NO: 33 - DNA, 20 bp, Artificial sequence
  Description of the artificial sequenceOligonucleotide
accgttttccatgagcaaac

SEQ ID NO: 34 - DNA, 20 bp, Artificial sequence
  Description of the artificial sequenceOligonucleotide
cccgtcctgtggatcctcta

SEQ ID NO: 35 - DNA, 20 bp, Artificial sequence
  Description of the artificial sequenceOligonucleotide
gttgaaagtgagacctcggc

SEQ ID NO: 36 - DNA, 20 bp, Artificial sequence
  Description of the artificial sequenceOligonucleotide
tgccacaaatgttaaggcaa

SEQ ID NO: 37 - DNA, 19 bp, Artificial sequence
  Description of the artificial sequenceOligonucleotide
aaaacgacggccagtgagc

SEQ ID NO: 38 - DNA, 21 bp, Artificial sequence
  Description of the artificial sequenceOligonucleotide
ttttaccgacatggtacctgg

SEQ ID NO: 39 - DNA, 24 bp, Artificial sequence
  Description of the artificial sequenceOligonucleotide
aagctcactggccgtcgttttaaa

(19) United States
(12) Patent Application Publication
(10) Pub. No.: US 2005/0196756 A1
(43) Pub. Date: Sep. 8, 2005

(54) Method for the detection of nucleic acid molecules

(76) Inventors: Wolfgang Schmidt, Vienna (AT); Axel Mundlein, Vienna (AT); Martin Huber, Vienna (AT); Hans Kroath, Baden (AT)

Correspondence Address: FULBRIGHT & JAWORSKI L.L.P., 600 CONGRESS AVE., SUITE 2400, AUSTIN, TX, 78701, US

(21) Appl. No.: 10/469,713

(22) PCT Filed: Mar. 1, 2002
(86) PCT No.: PCT/AT02/00060

§ 371 Date: Jul. 12, 2004

(30) Foreign Application Priority Data
Mar. 2, 2001 (AT) A3372001

(51) Int. Cl. C12Q 1/68; C12P 19/34

(52) U.S. Cl. 435/6; 435/91.2

(57) ABSTRACT

There is provided a method of simultaneously detecting at least two mutually different nucleic acid molecules in a sample, wherein in a first step a multiplex-PCR and in a second step a hybridizing reaction is carried out with probes immobilized on a microarray, whereupon the hybridized PCR products are detected and optionally quantitated, wherein the probes employed for the hybridizing reaction which in each case will hybridize specifically with the mutually different nucleic acid molecules have melting temperatures which differ from each other by 2° C. at the most, preferably by 1° C. at the most.

The present invention relates to a method of simultaneously detecting at least two mutually different nucleic acid molecules in a sample, wherein in a first step a multiplex PCR and in a second step a hybridizing reaction is carried out with probes immobilized on a microarray, whereupon the hybridized PCR products are detected and optionally quantified, as well as a microarray and a set for hybridizing multiplex-PCR products, and a kit for the simultaneous detection of at least two mutually different nucleic acid molecules in a sample.

The detection of nucleic acid molecules in a sample is carried out in the most various areas, e.g. in medicine, in quality check-ups, in research etc. Often it is necessary to detect at least two mutually different nucleic acid molecules, often 20, 50, 100 or more, in a sample. For reasons of time and costs it is desirable to detect the different nucleic acid molecules simultaneously in one sample. A series of publications relate to the detection of nucleic acid molecules and disclose various methods for carrying out the detection:

In U.S. Pat. No. 5,994,066, a method for detecting bacterial or antibiotic resistances, respectively, in biological samples is described. According to a first method, a multiplex-PCR is carried out for the simultaneous detection of several antibiotic resistances. As an example of the detection of the amplified products, agarose gel electrophoresis, fluorescence polarization and the detection by means of fluorescence labeling have been mentioned. A hybridization method is described as a further, second method of detecting the sequences searched for in samples, hybridization being carried out at 65° C., and the hybridization of a sample with the specific target DNA indicating a high degree of identity between the two nucleotide sequences.

U.S. Pat. No. 6,045,996 describes a method for hybridizing a nucleotide sequence on a microarray. Temperatures of between 20 and 75° C. are indicated as the hybridization temperature. As an example of target nucleotides, amplification products of a multiplex PCR are mentioned.

According to U.S. Pat. No. 5,614,388, specific nucleotide sequences are amplified by means of PCR, whereupon the amplification products are detected by hybridizing. As the preferred embodiment, a multiplex PCR is carried out. The detection may be specifically carried out by adjusting stringent conditions. As stringent hybridizing conditions, temperatures are stated which allow for a specific hybridization. As an example of a hybridizing temperature, 50 to 55° C. are indicated.

U.S. Pat. No. 5,846,783 relates to a method of detecting nucleotide sequences, wherein following a multiplex PCR, a detection by means of hybridizing is carried out. For example, the hybridization is carried out at a temperature of 55° C.

WO 98/48041 A2 relates to a method for identifying antibiotic-resistant bacterial strains, wherein the genes are amplified via PCR and detected by means of hybridizing probes. In doing so, hybridizing is to be carried out under stringent conditions, such as 20° C. below the melting point of the hybridizing DNA. The oligonucleotides preferably are chosen such that they have similar melting temperatures and thus several genes in the same hybridizing mixture can be tested by the same conditions. As an example, furthermore, the hybridization on an oligonucleotide microarray is described. As the hybridizing temperature, a temperature of from 45 to 60° C. is indicated.

However, all these above-mentioned methods have the disadvantage that there are limits as regards the specificity and the maximum number of nucleic acid molecules that are simultaneously detectable. In some of these methods, a multiplex PCR is carried out in a first step, whereby the simultaneous amplification of several nucleotide sequences is ensured. The subsequent detection of the various nucleotide sequences is, however, a problem, since according to this method it is not possible to simultaneously specifically detect a larger number of nucleotide sequences. If a hybridizing reaction is carried out after the PCR reaction, specific, stringent hybridizing conditions must be adjusted for each nucleotide sequence, a lower temperature being adjusted for shorter sequences than for longer sequences, cf. e.g. U.S. Pat. No. 6,045,996, whereby, however, the possible number of simultaneously detectable nucleotide sequences decreases. In WO 98/48041 A2, it has, e.g., been suggested to select oligonucleotides which have similar melting temperatures so that several genes can be tested in the same hybridizing mixture, wherein, however, a maximum of eight oligonucleotides is tested on one array.

Thus, these methods are not suitable to carry out methods for the detection of several or a large number of nucleic acid molecules, e.g. for the detection of antibiotic resistances. For such detection methods, a method which is restricted to a simultaneous detection of merely a few oligonucleotides is insufficient and too labor intensive and time-consuming in practice, in particular for screens.

Therefore, the present invention has as its object to provide a method in which a large number of nucleic acid molecules can be detected simultaneously, so that a detection of certain oligonulceotides or genes, respectively, in a sample can be carried out quickly, cost-efficiently and with little work involved.

The initially indicated method of the present invention is characterized in that the probes employed for the hybridizing reaction which in each case will hybridize specifically with the mutually different nucleic acid molecules have melting temperatures (Tₘ) which differ from each other by 2° C. at the most, preferably 1° C. at the most. By the fact that the melting temperatures of the probes used for the hybridizing reaction differ from each other by 2° C. at the most, or preferably, by 1° C., at the most, it has become possible for the first time to simultaneously detect a large number of nucleic acid molecules in one sample, since the same conditions as regards temperature and also salt concentration, pH, etc., will be adjusted for the hybridizing reaction for all the probes. The melting temperature Tₘ is defined as that temperature at which (under given parameters, such as, e.g., salt concentration), half of all the molecules will be in the helical state.

It is possible to provide sequences with a certain melting temperature for nearly all nucleic acid molecules:

One possible way of calculating the melting temperature of a sequence is by means of the commercial software “Gene Runner 3.0” (© 1994, Hastings Software, Inc.). This software allows the Tₘₛ to be determined by means of various methods/algorithms. The statements in the present patent application are values of the so-called “nearest-neighbor thermodynamic melting temperature”-method according to Breslauer et al. (Proc. Natl. Acad. Sciences 83: 3746-3750, Predicting DNA duplex stability from the base sequence). The parameters for the calculation may, e.g. be 660 mM for the salt concentration and 7.5 pM for the sample concentration. For determining the Tₘₛ of several probes for a simultaneous hybridizing experiment, it is not the absolute values (which may be higher or lower, depending on salt and DNA concentration) which are decisive, but the method chosen (i.e. for probes having a length of between 15 and 30 bases, the “thermodynamic one”) and the values for the Tₘₛ of the individual probes in relationship relative to each other. In this manner, the sequence to be hybridized, “hybridizing sequence”, for the nucleic acid molecules or genes, respectively, to be tested can be calculated and chosen so that specific probes therefor can be prepared.

Within the scope of the present invention, by nucleic acid molecules, portions of sequences are to be understood which are, e.g., certain genes, parts of a gene or genome, an mRNA or parts of an mRNA, etc.

By the term “multiplex-PCR” within the scope of the present invention, a PCR is to be understood in which simultaneously at least two mutually different nucleic acid molecules are amplified, i.e. that with the assistance of different primers, different sequences can be amplified simultaneously in one reaction.

By “microarray” a carrier is to be understood on which a high number of probes are immobilized in high density so that under the same conditions, simultaneously a large number of nucleic acid molecules can be hybridized. Microarrays usually are used for the detection of DNA molecules, yet microarrays already are also being used for the detection of peptides. With the assistance of microarrays, the in vitro DNA-diagnosis has been substantially simplified so that complex tests can be carried out very rapidly in one single working step, since several thousands of specifically designed oligonucleotides can be immobilized on the relatively small microarrays. For instance, the hybridization on a microarray ensures the simultaneous examination of tens of thousands of genes. A series of different microarrays have already been used for the detection of nucleiotide sequences, the different parameters being chosen by the person skilled in the art in a wide range (cf. e.g., Lockhart et al., Nature Biotechnology, vol. 14, December 1996, pp. 1675-1679).

Within the scope of the present invention, it is e.g., possible to adapt and vary the material, size, structure etc. of the microarray to the probes to be immobilized as regards the number, length and sequence thereof.

On the one hand, it is possible to merely detect the nucleic acid molecules, i.e. to test whether or not they are present in a sample, and this test will yield a YES/NO result. According to the invention, however, it is also possible to quantify the amount of the nucleic acid molecules in the sample, and this can be carried out highly specifically because of the use of the microarrays. For this, any detection method known to the person skilled in the art may be used, e.g., chemical, enzymatic, physico-chemical or antigen-antibody binding processes may be employed. The nucleic acid to be detected can be labeled, e.g. with a radioactive, fluorescent or chemoluminescent molecule. These detection methods are very well known to the person skilled in the art and therefore need not be discussed here in detail, the choice of the respective method depending on the nucleic acid molecules to be detected and on whether the product is merely to be detected or to be quantified.

The preparation of the probes is effected according to methods known per se.

The less the melting temperatures of the probes differ from each other, the more specific the nucleic acid molecules can be detected since by this hybridization conditions can be adjusted which will merely ensure a highly specific hybridization, yet not a hybridization of not completely complementary sequences, whereby the risk of the falsely positive, but also of falsely negative results is lowered or completely eliminated.

The primer and probes can be chosen such that nucleic acid molecules are amplified which have a sequence longer than the hybridizing sequence, i.e. that sequence which hybridizes with the probes. It is however, also possible that merely the hybridizing sequence is amplified, i.e. that the nucleic acid molecule only consists of that sequence with which the respective probes hybridize.

Preferably, according to the invention at least 6, preferably at least 8, particularly preferred at least 12 nucleic acid molecules which differ from each other are simultaneously detected in a sample. The number of mutually different nucleic acid molecules detected in the sample will depend on the specific case, there being practically no upward limits.

Particularly preferably, nucleic acid molecules are detected which are contained in antibiotic resistance genes. A large number of antibiotic resistance genes is known, the detection methods as a rule being carried out by long and error-prone microbiological growth tests on antibiotic-containing nutrient media and subsequent determination of the viable germs. Even though methods for the identification of antibiotic resistances with the assistance of gene amplifications and subsequent hybridizing have already been described (cf. WO 98/48041 A2), it has not been possible to test one sample for several antibiotic resistance genes simultaneously, without a reduction of the specificity. With the method according to the invention it has now become possible to detect an unlimited number of antibiotic resistance genes in a sample, which is of particular importance in the field of hospitals since an accumulation of antibiotic-resistant bacterial strains will occur there. All the standard DNA isolation methods are functional. In any event, it should be ensured that smaller molecules (such as plasmids, e.g.) are copurified so as not to lose episomally encoded resistances.

As the nucleic acid molecules, parts of sequences from the antibiotic resistance genes are chosen which are specific of the respective gene and do not occur in other genes. In this manner, falsely positive test results can be even better prevented.

Preferably, the antibiotic resistance genes are selected from the group consisting of genes for the beta-lactamase blaZ, chloramphenicol acetyltransferase, the fosB protein, the adenin methylase ermC, aacA-aphD aminoglycoside resistance, 3′5′-aminoglycoside phosphotransferase aphA-3, mecR, the penicillin binding protein PBP2′, the aminoglycoside-3′-adenyltransferase aada, the tetracycline-resistance protein tetc, DHFR DfrA and the D-Ala:D-Ala ligase vanB. These are frequently occurring antibiotic resistances which cause severe medical difficulties, and thus it is particularly important for these antibiotic resistances to provide a rapid and highly specific test method. It is particularly suitable if all these said antibiotic resistances can be tested simultaneously in one sample, i.e. that the nucleic acid molecules which are respectively specific of each of these antibiotic resistance genes are simultaneously amplified in a multiplex PCR and subsequently hybridize with probes on a microarray, wherein at least one probe each is specific for a nucleic acid molecule and thus, for an antibiotic resistance gene.

It is particularly suitable if the hybridizing reaction is carried out at 30-80° C., preferably at 40-70° C., particularly preferred at 55-65° C. The hybridizing temperature to be adjusted is dependent on the melting temperature of the probes and, according to the invention, may be calculated and adjusted for each hybridizing reaction, it being particularly important that the temperature be held constant during the hybridizing reaction. It has been shown that it is particularly suitable for the present method to adjust temperatures of between 55 and 65° C., since in this temperature range probes have melting temperatures which are particularly well suited for the present method, in particular as regards specificity and length.

For a particularly precise detection, it is advantageous if the hybridizing reaction is carried out under highly stringent conditions. This means that hybridizing conditions are adjusted which will ensure a hybridizing of highly complementary sequences, yet not of sequences which differ in a few nucleotides. It is particularly advantageous if hybridizing conditions are chosen under which only completely complementary sequences will bind to each other, yet not sequences which differ merely in one single nucleotide. In this manner, a method is provided which ensures a highly specific detection of nucleic acid molecules in a sample and which will not give false positive results. The highly stringent conditions are adjusted by choosing the temperature and ionic strength in the reaction mixture. For instance, the hybridizing temperature is adjusted to 5 to 100 below the melting temperature of the probes; the buffer(s) will be chosen according to the desired ionic strength or pH in dependence on the hybridizing temperature.

Preferably, the multiplex-PCR is carried out with primers that are labeled. In this manner, it is ensured that the amplified PCR products will have a labeling that can be detected after the hybridizing reaction. As has already been described above, the labeling may consist in a molecule, a chemically, physico-chemically or enzymatically detectable signal, which can be determined and quantified, e.g., via a color reaction by measuring the fluorescence, luminescence, radioactivity etc.

For a particularly specific method it is suitable if the hybridizing reaction is carried out after separation of the “+” and “−” strands. Thereby it is avoided that the strands which have a sequence identical to the probes will competitively bind with these probes to the individual strand molecules to be detected, which would lead to falsified results particularly in case of a quantitative detection. By separating the “+” and the “−” individual strands, merely the individual strands complementary to the probes will be present in the hybridizing mixture.

In doing so, it is particularly advantageous if the “+” individual strands which have sequences identical to the probes are separated after the multiplex-PCR. In this manner, the “−” individual strands which have sequences complementary to the probes will remain in the hybridizing mixture so that the hybridizing reaction can be carried out immediately thereafter.

A particularly advantageous separating procedure is characterized in that primers are used for the elongation of the “+” individual strands which, preferably at their 5′ terminus, each are coupled to a substance, in particular at least one biotin molecule, which ensures the separation of the “+” individual strands. In this manner, the “+” individual strands can be changed already in the amplification step of the PCR so that their complete separation will be specifically ensured without having to incorporate additional intermediate steps into the method. In this manner, the risk that also the “−” individual strands will be separated is eliminated. Biotin is particularly suitable since it can easily be coupled to a DNA sequence and can be separated specifically.

For this purpose, it is particularly suitable if biotin molecules are coupled to the primers for the elongation of the “+” individual strands, the “+” individual strands being separated after the multiplex-PCR by means of streptavidin bound to beads. By means of the beads it is made possible that a large area of streptavidin is introduced into the sample, whereby the biotin molecules will completely bind to the streptavidin. Furthermore, by using the beads it is ensured that the streptavidin-biotin compound will be separated again from the sample. The beads used therefor are known per se and may, e.g., be made of glass or with a magnetic core, respectively.

Preferably, a purification step precedes the hybridizing step. In this manner substances which possibly could interfere in the hybridization are removed from the hybridizing mixture, this purification step optionally occurring during or after the separation of the “+” individual strands. The purification may, e.g., be carried out by precipitation of the DNA and re-uptake of the DNA in a buffer.

According to a further aspect, the present invention relates to a microarray for hybridizing multiplex-PCR products according to any one of the above-described inventive methods, wherein at least two, preferably at least six, particularly preferred at least twelve probes which each specifically hybridize with the mutually different nucleic acid molecules to be detected, are bound to its surface and have melting temperatures which differ from one another by 2° C. at the most, preferably by 1° C. at the most. As regards the microarray and the probes, the definitions already set out above for the method also apply here. Again, the number of probes bound to the microarray will depend on the number of the nucleic acid molecules to be detected, wherein, of course, also additional probes which do not hybridize with the nucleic acid molecules to be detected may be bound to the microarray as a negative test. What is important is, as has already been described above, that the melting temperatures of the probes differ from one another by merely 2° C. at the most, preferably by 1° C. at the most, whereby it is ensured that conditions can be adjusted for the hybridizing reaction under which all the nucleic acid molecules which have a sequence that is complementary to the probes will hybridize equally specifically and tightly with the probes.

Preferably, the probes are bound to the surface of the microarray in spots having a diameter of from 100 to 500 μm, preferably from 200 to 300 μm, particularly preferred 240 μm. It has been found that spots having this diameter are particularly well suited for the above-described method according to the invention, a detection following the hybridizing reaction yielding particularly clear and unmistakable results. One spot each exhibits one type of probe, i.e. probes having the same sequence. It is, of course, also possible to provide several spots with the same type of probe on the microarray, as parallel tests.

Furthermore, it is advantageous if the spots have a distance from each other of from 100 to 500 μm, preferably from 200 to 300 μm, particularly preferred 280 μm. In this manner it will be ensured that a maximum number of spots is provided on the microarray, it being possible at the same time to clearly distinguish in the detection procedure between the various spots and, thus, probes and bound nucleic acid molecules to be detected.

Preferably, the microarray is made of glass, a synthetic material or a membrane, respectively. These materials have proven particularly suitable for microarrays.

It is particularly suitable if the probes are covalently bound to the surface of the microarray. In this manner, a tight bond of the probes to the microarray will be ensured without a detachment of the probe-microarray bond and, thus, a falsified result occurring in the course of the hybridizing and washing steps. If the microarray is made of coated glass, e.g., the primary amino groups can react with the free aldehyde groups of the glass surface under formation of a Schiff's base.

It has proven to be suitable if the probes have a hybridizing sequence comprising 15 to 25, preferably 20, nucleotides. By hybridizing sequence, as has already been described above, that sequence is to be understood with which the nucleic acid molecules to be detected will hybridize. Of course, the probes may be made longer than the hybridizing sequence, yet with the increase in the additional length of the probe, an undesired bond with other nucleic acid molecules could occur, which would falsify the result. Therefore, it is advantageous if the probes—besides the parts which are required for the binding to the surface of the microarray—merely consist of the hybridizing sequence. The length of from 15 to 25, preferably 20, nucleotides has proven suitable since in this length range it is possible to find hybridizing sequences with the above-described methods, which have the required melting temperature. This length is sufficient to allow for a specific binding and to eliminate the risk that also other DNA molecules by coincidence have the same sequence as the nucleic acid molecules to be detected.

Preferably, the probes at their 5′ terminus each have a dT10 sequence via which they can be bound to the microarray. In this manner, the distance between the microarray and the hybridizing sequence will be sufficient so that the latter will be freely accessible to the nucleic acid molecules. The number of the Tₘ may, e.g., be from five to fifteen, preferably ten.

For the simultaneous detection of antibiotic resistance genes it is suitable if as the hybridizing sequence, the probes comprise a sequence selected from the group consisting of No. 25, No. 26, No. 27, No. 28, No. 29, No. 30, No. 31, No. 32, No. 33, No. 34, No. 35 and No. 36. These sequences occur in antibiotic resistance genes which especially frequently occur in bacterial strains and are medically important. These are the antibiotic resistance genes for the beta-lactamase blaZ, chloramphenicol acetyltransferase, the fosB protein, the adenin-methylase ermC, aacA-aphD aminoglycoside resistance, 3′5′-aminoglycoside phosphotransferase aphA-3, mecR, the penicillin binding protein PBP2′, the aminoglycoside-3′-adenyltrasnferase aada, the tetracycline-resistance protein tetC, DHFR DfrA and the DAla:D-Ala ligase vanB and have melting temperatures which differ from one another by about 1° C. at the most.

According to a further aspect, the present invention relates to a set for hybridizing multiplex-PCR products according to any one of the above-described methods of the invention, which set comprises at least two, preferably six, particularly preferred at least twelve probes, each specifically hybridizing with the mutually different nucleic acid molecules to be detected and having melting temperatures that differ from each other (i.e. from the respective other probe molecules/detected nucleic acid pairs in the set) by 2° C. at the most, preferably by 1° C. at the most. The probes may be dissolved in a buffer. Furthermore, the set may comprise several containers, probes with the same sequence being present per container. By this it will be possible to apply probes of the same sequence on the microarray per spot. It is, of course, also possible to provide probes with two or more sequences that differ from each other in one container.

Preferably, the probes have a hybridizing region comprising 15 to 25, preferably 20, nucleotides.

Furthermore, it is suitable if the probes each have a dT sequence at their 5′ terminus, the number of the Tₘ preferably being between 5 and 15, e.g. 10.

It is particularly advantageous if the probes in their hybridizing region each have a sequence which is selected from the group consisting of No. 25, No. 26, No. 27, No. 28, No. 29, No. 30, No. 31, No. 32, No. 33, No. 34, No. 35 and No. 36.

According to another aspect, the present invention relates to a kit for simultaneously detecting at least two mutually different nucleic acid molecules in a sample, the kit comprising
a microarray according to the invention, as described above,
at least one container with primers for the specific amplification of the nucleic acid molecules to be detected and
optionally, a set according to the invention as described above. A container may comprise primers with the same sequence, but also a primer pair for amplification of a nucleic acid molecule, or finally also several primer pairs for the amplification of several mutually different nucleic acid molecules, wherein, however, the primers should be present at a certain concentration. The kit may, of course, also further comprise user's instructions with a protocol for carrying out the above-described inventive method, as well as possible further buffers, salts, solutions etc. which are necessary for the amplification reaction, hybridizing reaction, and detection, respectively.

The microarray may comprise probes already immobilized thereon. The set comprising the probes may be present separate from the microarry (in case that the microarray is blank, i.e. that it does not contain any bound probes), yet it may also be an integrated component of the microarray.

Preferably, the kit further comprises at least one container with at least one nucleic acid molecule to be detected, as positive sample. Also here, a container again may comprise nucleic acid molecules with the same sequence, it being possible that several containers are provided in the kit, yet it is also possible to provide nucleic acid molecules with several, mutually different sequences in one container. For instance, if the kit is provided for the detection of antibiotic resistance genes, the kit may provide nucleic acid molecules with the sequences with the hybridizing sequence SEQ ID No. 25 to SEQ ID No. 36, as a positive sample.

It is particularly suitable if the kit further comprises a container with streptavidin bound to beads. This allows for a separation of the amplified “+” and “−”individual strands, if the “+” or “−” individual strand is coupled to biotin, e.g. by using primers coupled to biotin.

In the following, the invention will be explained in more detail by way of the example given as well as by way of the figures to which, however, it shall not be restricted.

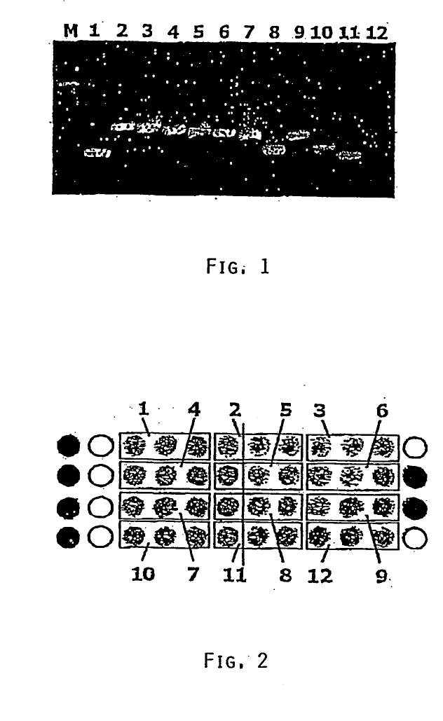

FIG. 1 shows the separation of the PCR products of all twelve ABR targets by means of gel electrophoresis;

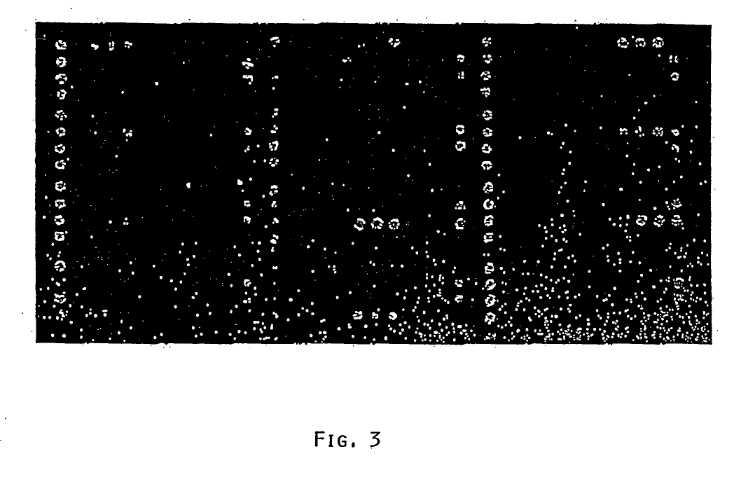

FIG. 2 shows the microarray layout of the ABR chip;

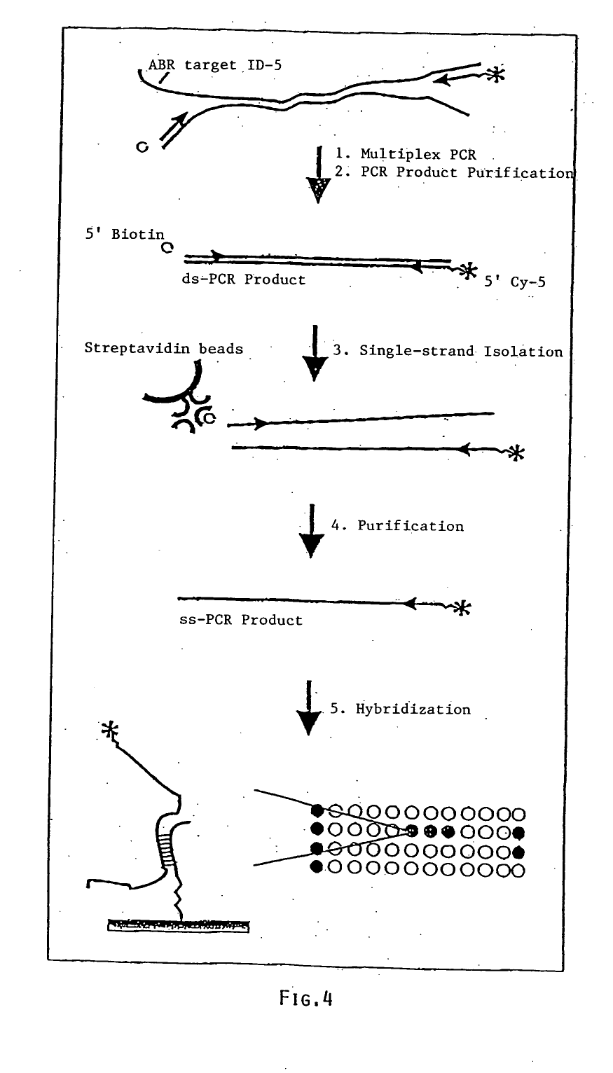

FIG. 3 shows the diagram of the test course;

FIG. 4 shows an illustration of the control hybridization on the ABR chip; and

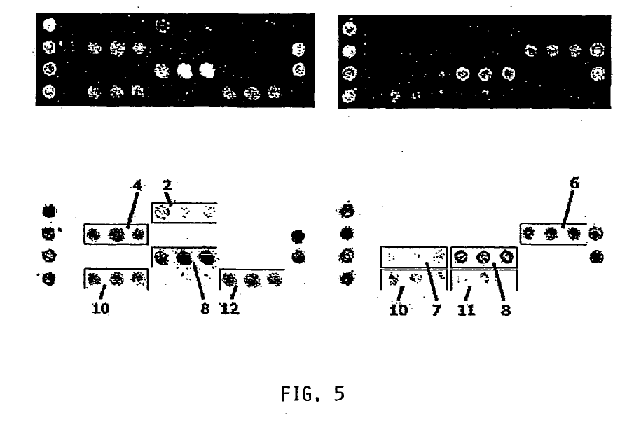

FIG. 5 shows the result of the ABR chip detection after the multiplex amplification.

### EXAMPLE

1. Gene Synthesis of Reference “ABR Targets”

A series of antibiotic resistance (ABR) sequences was prepared by gene synthesis in vitro, since either “type strains” were not available or working with the organisms in question was not possible for safety reasons (bio-safety level 2 or higher). In Table 1 all the targets are summarized and provided with a number (No.), the control resistances being derived from vectors and primarily serving to validate the chip. For these targets, probes are provided on the ABR chip prototype.

TABLE 1

| | Antibiotic | | Target |
|---|---|---|---|
| No. | Resistance | Species | (resistance gene) |
| 8 | Ampicillin | S. aureus, E. faecalis | beta-lactamase blaZ |
| | (control) | | |
| 9 | Chloram- | Bacillus sp., | Chloramphenicol acetyl- |
| | phenicol | Corynebacterium sp. | transferase |
| | (control) | | |
| 11 | Fosformycin | S. epidermidis, | fosB protein |
| | | Staphylococcus sp. | |
| 7 | Erythromycin | Staphylococcus sp. | Adenin methylase ermC |
| 12 | Gentamycin | S. aureus | aacA-aphD Aminoglyco- |
| | | | side resistance gene |
| 2 | Kanamycin | S. aureus, S. faecalis, | 3′5′-Aminoglycoside |
| | | E. faecalis | phosphotransferase |
| | | | aphA-3 |
| 3 | Methicillin | S. aureus | mecR |
| 1 | Penicillin | S. aureus | Penicillin binding protein |
| | | | PBP2′ |
| 5 | Streptomycin, | Salmonella | Aminoglycoside-3′- |
| | Spectinomycin | | adenyltransferase |
| | | | aadA |
| 10 | Tetracycline | Salmonella sp. | Tetracycline resistance |
| | (control) | | protein tetC |
| 4 | Trimethoprim | S. aureus | DHFR DfrA |
| 6 | Vancomycin- | Enterococcus, | D-Ala:D-Ala ligase vanB |
| | VanB-Type | Streptococcus | |

Table 2 gives the sequences of the PCR primers and the lengths of the PCR products which were developed for the prototype, in FIG. 1 all 12 PCR products after agarose gel electrophoresis can be seen.

TABLE 2

| No. | Name | PCR Primer (SEQ ID No.) | PCR Product |
|---|---|---|---|
| 1 | PBP2 | 1 + 2 | 423 bp |
| 2 | KanR | 3 + 4 | 532 bp |
| 3 | MecR | 5 + 6 | 517 bp |
| 4 | DhfrA | 7 + 8 | 279 bp |
| 5 | StrR | 9 + 10 | 549 bp |
| 6 | VanB | 11 + 12 | 498 bp |
| 7 | MlsR | 13 + 14 | 564 bp |
| 8 | AmpR | 15 + 16 | 219 bp |
| 9 | CmR | 17 + 18 | 247 bp |
| 10 | TetR | 19 + 20 | 245 bp |
| 11 | FosB | 21 + 22 | 304 bp |
| 12 | AacA | 23 + 24 | 497 bp |

2. Microarray Design

For each available (PCR) nucleic acid molecule of the antibiotic resistance (ABR) genes in question, highly specific DNA probes were located by means of bioinformatic standard methods. Generally, the software “Gene Runner 3.0” (© 1994, Hastings Software, Inc.) was used, for the PCR and Hyb Primer selection “Primer 3”, Steve Rozen & Helen J. Skaletsky (1998) Primer 3 was used, for homology search and database cross-checks “Fasta3”, W. R. Pearson and D. J. Lipman (1988), “Improved Tools for Biological Sequence Analysis”, PNAS 85:2444-2448, W. R. Pearson (1990), “Rapid and Sensitive Sequence Comparison with FASTP and FASTA” Methods in Enzymology 183:63-98 was used, for alignments “ClustalX 1.8”, Thompson, J. D., Gibson, T. J., Plewniak, F., Jeanmougin, F. and Higgins, D. G. (1997), The ClustalX windows interface: flexible strategies for multiple sequence alignment aided by quality analysis tools. Nucleic Acids Research, 24:4876-4882, was used. Particular attention was paid to the fact that potential cross-hybridizations with other possible ABR targets can be excluded. Extensive EMBL and GenBank database searches were employed so as to make sure that the respective probes do not allow hybridizations in error with “foreign” sequences. The probes are localized in A/T rich regions of the PCR fragments so as to ensure optimum conditions during hybridization with dsPCR products. Optimum conditions in this instance mean that hybridizations are generally more efficient if the probe “recognizes” a region in the dsDNA which denatures more easily (because, e.g., in a region richer in A/T.

Each probe has a Tₛ value of 65° C.±1 and has an extra dT₁₀ sequence at the 5′ terminus as a spacer between the chip surface and the hybridizing sequence (cf. Table 3). All the oligonucleotides were synthesized with a 5′ (CH₂)₆—NH₂ modification and purified by means of a reversed phase chromatography HPLC protocol. The probes are adjusted to a concentration of 1 mM and stored at −20° C. in MT plates.

TABLE 3

| | | Sequence (SEQ ID | |
|---|---|---|---|
| No. | Name | No.) | Tₘ |
| 1 | PBP2 | 25 | 64.8° C. |
| 2 | KanR | 26 | 65.1° C. |
| 3 | MecR | 27 | 64.8° C. |
| 4 | DhfrA | 28 | 65.5° C. |
| 5 | StrR | 29 | 63.7° C. |
| 6 | VanB | 30 | 64.4° C. |
| 7 | MlsR | 31 | 64.9° C. |
| 8 | AmpR | 32 | 65.4° C. |
| 9 | CmR | 33 | 64.9° C. |
| 10 | TetR | 34 | 65.9° C. |
| 11 | FosB | 35 | 64.2° C. |
| 12 | AacA | 36 | 65.0° C. |
| Co | BSreverse | 37 | 65.9° C. |
| hCo | AT-M33 | 38 | 65.3° C. |

3. Array Layout

The probes are covalently bound to the glass surface, face, and in doing so, the 5′ primary amino groups react with free aldehyde groups of the glass surface under formation of a Schiff's base (“Silylated Slides”, CEL Associates). The probes were applied to the glass carriers by means of a spotter (Affymetrix 417 Arrayer). In doing so, the spotting protocols were optimized for a good reproducibility and spot consistence. Spotting was effected in 3×SSC 0.1% SDS with hits/dot. The spots have a diameter of approximately 240 μm and are applied on the microarray with a spotto-spot distance of 280 μm. There exist two replicas for each spot. For validating the chip, control probes (Bluescript polylinker sequence) are applied in a typical pattern (“guide dots”) and negative controls (blank values, so-called “buffer dots”).

FIG. 2 shows an array layout of the ABR chip. The position of the 12 ABR targets is denoted with the respective numbers (No.). “Guide dots” are black, “buffer dots” are white, the position of the heterologous controls is marked in gray.

4. Chip Validation and Control Hybridization

The hybridizing conditions are mainly optimized on the microarray with the help of the control probe set. Six spots on the microarray contain a control probe with a BS polylinker-specific sequence. Hybridization was carried out in a 7 μl volume with a 3′ terminal Cy5-dCTP labeled oligonucleotide (BSrevco, 5′ AAGCTCACTGGCCGTCGTTTTAAA SEQ ID No. 39) in SSARC buffer under a 15×15 mm (2.25 mm²) cover slip for 1 hour at 55° C.

The chip was washed according to standard protocols (2×SSC 0.1% SDS, then 0.2×SSC 0.1% SDS, then with 0.2×SSC and finally with 0.1×SSC, 2 min each). Then the glass carrier was scanned in a confocal fluorescence scanner (Affymetrix 418 Array Scanner) with a suitable laser output and suitable PMT voltage adjustments.

In FIG. 3, the control hybridization on the ABR chip is shown. The result of a total of 12 individually effected hybridizing experiments with one of the specific targets each (No. 1 to no. 12) with the “guide dot” controls (from left to right in each case No. 1 to No. 3, No. 4 to No. 6, No. 7 to No. 9 and No. 10 to No. 12) can be seen.

5. Pilot Studies with the ABR Chip Prototype

In a first functional test of the ABR chip, two different sample mixtures (“MixA” and “MixB”) of three different ABR targets each and two control targets (Ampicillin No. 8 and Tetracycline No. 10) were prepared: “MixA” contained kanamycin (aphA-3 No. 2), trimethroprim (dhfrA No. 4) and gentamycin (aacA No. 12) in addition to the control targets, “MixB” contained vancomycin (vanB No. 6), erythromycin (ermC No. 7) and fosfomycin (fosB No. 11) in addition to the control targets. The synthetic templates were used so as to allow for as exact an adjustment of the template amounts as possible. The multiplex amplification was carried out under standard PCR conditions in 35 cycles, wherein the primers for the amplification of the “+” individual strands which are identical to the probes were coupled to biotin molecules. The primers for the “−” individual strands which have sequences complementary to the probes were coupled to marker molecules 5′Cy-5: The reaction formulation was purified, individual strands were isolated by means of alkaline denaturing on dynabeads and hybridized in SSARC buffer for 1 hour at 55° C. on the ABR prototype arrays (cf. FIG. 4):

A) PCR (Controls in the Individual Formulation)
25 μl Volume:
   1.0 μl template (3 fmol/μl)
   1.0 μl primer plus (25 μM) 5′-biotin [VBC-GENOMICS]
   1.0 μl primer minus (25 μM) 5′-Cy5 [VBC-GENOMICS]
   2.5 μl HotStar™ buffer (10×) [Qiagen]
   0.5 μl dNTPs (10 mM) [Roche]
   0.1 μl HotStar™ Taq DNA polymerase [Qiagen]
   ad 25 μl with aqua bidest.
Cycling: 15 min 95° C. 30×[30 sec 95° C. 20 sec 60° C. 40 sec 72° C.] 10 min 72° C.
Purification of the PCR formulation by means of QIA-quick™ PCR-Purification Kit

B) Multiplex-PCR
50 μl Volume:
   ×μl template (×fmol/μl)
   6.0 μl primer “cocktail” plus (je 25 μM) 5′-biotin [VBC-GENOMICS]
   6.0 μl primer “cocktail” minus (je 25 μM) 5′-Cy5 [VBC-GENOMICS]
   5.0 μl HotStar™ buffer (10×) [Qiagen]
   1.0 μl dNTPs (10 mM) [Roche]
   0.2 μl HotStar™ Taq DNA polymerase [Qiagen]
   ad 50 pl with aqua bidest.
Cycling: 15 min 95° C. 35×[30 sec 95° C. 20 sec 55° C. 40 sec 72° C.] 10 min 72° C.
Purification of the PCR formulation by means of QIAquick™ PCR Purification Kit

C) Single-Strand Isolation
Wash 20 μl Dynabeads (10 μg/μl) [Roche] 2× with 200 μl 1×TS buffer
take up in 8 μl 6×TS buffer
incubate with 40 μl of the PCR formulation for 30 min at 37° C.
wash Dynabeads 2× with 200 μl 1×TS buffer
denature DNA 2× with 20 μl 0.2 N NaOH for 5 min at RT
precipitate with 120 μl 90% EtOH/0.3 M NaOAC (20 min −20° C., 30 min at 16,500 rpm 4° C.)
wash pellet with 70% EtOH, dry and take up in 14 μl SSARC buffer

D) Hybridizing
Denature 7 μl of hybridizing sample in SSARC buffer with 0.1 μl BSrevco-Cy5 (1 μM) for 3 min at 98° C. and put on ice immediately
hybridize for 1 h at 55° C., under a 15×15 mm cover slip
Wash slide (2×SCC 0.1% SDS 5 min RT, 0.2×SSC 5 min RT, 0.2×SSC 5 min RT, 0.1×SSC 2 min RT)
scan slide

E) Buffer
TS buffer: 100 mM Tris-Cl, pH 7.6, 150 mM NaCl;
autoclaved
SSARC buffer: 4×SSC, 0.1% (w/v) Sarkosyl; 0.2 μm filtered
SSC buffer: 20×: 3 M NaCl, 0.3 M trisodium citrate (dihydrate), pH 7.0 The chips were washed and scanned.

FIG. 5 shows a multiplex amplification and subsequent ABR chip detection of two different synthetic targets and two control targets, “MixA” on the left, “MixB” on the right. “False color” images of the fluorescent scan can be seen, each under the negative with the correct allocation of the ABR target. As can be seen in FIG. 5, a clear allocation of the correct target is possible in the two different sample mixtures. This shows that the simultaneous detection of 12 nucleic acid molecules according to the method of the invention yields unambiguous results.

1-27. (canceled)

28. A method of simultaneously detecting mutually different nucleic acid molecules in a sample comprising:
obtaining a sample comprising at least two mutually different nucleic acid molecules each with a mutually different nucleic acid sequence;
amplifying the at least two mutually different nucleic acid molecules in a multiplex-PCR reaction to obtain at least two mutually different amplified nucleic molecules, each comprising an amplified portion having the nucleic acid sequence of one of the at least two mutually different nucleic acid molecules;
hybridizing the amplified portions of the at least two mutually different amplified nucleic acid molecules with at least two mutually different probes immobilized on a microarray to obtain at least two mutually different hybridized amplified nucleic acid molecules with melting temperatures that differ by at most 2° C.; and
detecting the at least two mutually different hybridized nucleic acid molecules.

29. The method of claim 28, further comprising quantifying the mutually different hybridized nucleic acid molecules.

30. The method of claim 28, wherein each of the at least two mutually different probes comprises a hybridizing sequence adapted to hybridize, during use, to the amplified portion of one of the at least two mutually different amplified nucleic acid molecules to produce a hybridized amplified nucleic acid molecule having a melting temperature which differs from the melting temperature of one or more other hybridized amplified nucleic acid molecules by at most 2° C.

31. The method of claim 28, wherein the at least two mutually different hybridized amplified nucleic acid molecules have melting temperatures which differ by at most 1° C.

32. The method of claim 28, wherein at least six mutually different nucleic acid molecules are simultaneously detected in the sample.

33. The method of claim 32, wherein at least eight mutually different nucleic acid molecules are simultaneously detected in the sample.

34. The method of claim 33, wherein at least twelve mutually different nucleic acid molecules are simultaneously detected in the sample.

35. The method of claim 28, wherein the mutually different nucleic acid molecules are comprised in at least two antibiotic resistance genes.

36. The method of claim 35, wherein at least one of the antibiotic resistance genes is the gene for beta-lactamase blaZ, chloramphenicol acetyltransferase, fosB protein, adenin methylase ermC, aacA-aphD aminoglycoside resistance, 3′5′-aminoglycoside phosphotransferase aphA-3, mecR, penicillin binding protein PBP2′, aminoglycoside-3′-adenyltransferase aadA, tetracycline-resistance protein tetC, DHFR DfrA, or D-Ala:D-Ala ligase vanB.

37. The method of claim 28, wherein the hybridizing reaction is carried out at 30-80° C.

38. The method of claim 37, wherein the hybridizing reaction is carried out at 40-70° C.

39. The method of claim 28, wherein the hybridizing reaction is carried out at 55-65° C.

40. The method of claim 28, wherein the hybridizing reaction is carried out under highly stringent conditions.

41. The method of claim 28, wherein the multiplex PCR is carried out with labeled probes. 25332285.1

42. The method of claim 28, further comprising the separation of “+” and “−” individual strands of the at least two mutually different amplified nucleic acid molecules prior to the hybridizing step.

43. The method of claim 42, wherein “+” individual strands of the at least two mutually different amplified nucleic acid molecules that have sequences identical to the probes are separated after the amplifying step.

44. The method of claim 43, wherein primers employed for the elongation of the “+” individual strands are coupled to a substance that ensures the separation of the “+” individual strands.

45. The method of claim 44, wherein the primers are coupled to the substance at their 5′ termini.

46. The method of claim 44, wherein the substance is at least one biotin molecule.

47. The method of claim 46, wherein the “+” individual strands are separated by means of streptavidin bound to beads.

48. The method of claim 28, further comprising purifying the at least two mutually different amplified nucleic acid molecules before the hybridizing step.

49. The method of claim 48, wherein the purification step occurs before separation of “+” and “−” individual strands of the at least two mutually different amplified nucleic acid molecules.

50. The method of claim 48, wherein the purification step occurs after separation of “+” and “−” individual strands of the at least two mutually different amplified nucleic acid molecules.

51. A microarray adapted to, during use, hybridize to amplified portions of at least two mutually different amplified nucleic acid molecules, the microarray comprising at least two mutually different probes immobilized on the microarray, wherein each probe comprises a hybridizing sequence adapted to hybridize, during use, to the amplified portion of one of at least two mutually different amplified nucleic acid molecules to produce a mutually different hybridized amplified nucleic acid molecule having a melting temperature which differs from the melting temperature of one or more other mutually different hybridized amplified nucleic acid molecules by at most 2° C.

52. The microarray of claim 51, wherein each probe comprises a hybridizing sequence adapted to hybridize, during use, to the amplified portion of one of the at least two mutually different amplified nucleic acid molecules to produce a hybridized amplified nucleic acid molecule having a melting temperature which differs from the melting temperature of one or more other hybridized amplified nucleic acid molecule by at most 1° C.

53. The microarray of claim 51, further defined as comprising at least six probes.

54. The microarray of claim 53, further defined as comprising at least twelve probes

55. The microarray of claim 51, wherein the probes are adapted to hybridize to amplified portions of mutually different nucleic acid molecules comprised in at least two antibiotic resistance genes.

56. The microarray of claim 55, wherein at least one of the antibiotic resistance genes is the gene for beta-lactamase blaZ, chloramphenicol acetyltransferase, fosB protein, adenin methylase ermC, aacA-aphD aminoglycoside resistance, 3′5′-aminoglycoside phosphotransferase aphA-3, mecR, penicillin binding protein PBP2′, aminoglycoside-3′-adenyltransferase aadA, tetracycline-resistance protein tetC, DHFR DfrA, or D-Ala:D-Ala ligase vanB.

57. The microarray of claim 51, wherein the probes are bound to the surface of the microarray in spots having a diameter of from 100 to 500 μm.

58. The microarray of claim 57, wherein the probes are bound to the surface of the microarray in spots having a diameter of from 200 to 300 μm.

59. The microarray of claim 58, wherein the probes are bound to the surface of the microarray in spots having a diameter of about 240 μm.

60. The microarray of claim 57, wherein the spots are from 100 to 500 μm apart.

61. The microarray of claim 60, wherein the spots are from 200 to 300 μm apart.

62. The microarray of claim 61, wherein the spots are 280 μm apart.

63. The microarray of claim 51, further defined as made of glass, a synthetic material, or a membrane.

64. The microarray of claim 51, wherein the probes are covalently bound to the surface of the microarray.

65. The microarray of claim 51, wherein the hybridizing sequences comprise 15 to 25 nucleotides.

66. The microarray of claim 65, wherein the hybridizing sequences comprise 20 nucleotides.

67. The microarray of claim 51, wherein the probes are bound to the microarray via dT sequences at their 5′ termini.

68. The microarray of claim 51, wherein the hybridizing sequence of at least one probe comprises a sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36.

69. The microarray of claim 68, wherein the hybridizing sequence of each probe comprises a sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36.

70. A probe set comprising at least two mutually different probes, wherein each probe comprises a hybridizing sequence adapted to hybridize, during use, to an amplified portion of one of at least two mutually different amplified nucleic acid molecules to produce a hybridized amplified nucleic acid molecule having a melting temperature which differs from the melting temperature of one or more other mutually different hybridized amplified nucleic acid molecule by at most 2° C.

71. The probe set of claim 70, further defined as comprising at least six probes.

72. The probe set of claim 71, further defined as comprising at least twelve probes.

73. The probe set of claim 70, wherein the hybridizing sequences comprise 15 to 25 nucleotides.

74. The probe set of claim 70, wherein the hybridizing sequences comprise 20 nucleotides.

75. The probe set of claim 70, wherein the probes comprise dT sequences at their 5′ termini.

76. The probe set of claim 70, wherein the hybridizing sequence of at least one probe comprises a sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36.

77. The probe set of claim 76, wherein the hybridizing sequence of each probe comprises a sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36.

78. The probe set of claim 70, wherein the probes are adapted to hybridize to amplified portions of mutually different nucleic acid molecules comprised in at least two antibiotic resistance genes.

79. The probe set of claim 78, wherein at least one of the antibiotic resistance genes is the gene for beta-lactamase blaZ, chloramphenicol acetyltransferase, fosB protein, adenin methylase ermC, aacA-aphD aminoglycoside resistance, 3′5′-aminoglycoside phosphotransferase aphA-3, mecR, penicillin binding protein PBP2′, aminoglycoside-3′-adenyltransferase aadA, tetracycline-resistance protein tetC, DHFR DfrA, or D-Ala:D-Ala ligase vanB.

80. A kit for simultaneously detecting at least two mutually different nucleic acid molecules in a sample, wherein the kit comprises:
a microarray adapted to, during use, hybridize to amplified portions of at least two mutually different amplified nucleic acid molecules, the microarray comprising at least two mutually different probes immobilized on the microarray, wherein each probe comprises a hybridizing sequence adapted to hybridize, during use, to the amplified portion of one of at least two mutually different amplified nucleic acid molecules to produce a mutually different hybridized amplified nucleic acid molecule having a melting temperature which differs from the melting temperature of one or more other mutually different hybridized amplified nucleic acid molecules by at most 2° C.; and
at least one container with primers for the specific amplification of nucleic acid molecules to be detected via multiplex-PCR during use.

81. The kit of claim 80, further comprising a probe set comprising at least two mutually different probes, wherein each probe comprises a hybridizing sequence adapted to hybridize, during use, to an amplified portion of one of at least two mutually different amplified nucleic acid molecules to produce a hybridized amplified nucleic acid molecule having a melting temperature which differs from the melting temperature of one or more other mutually different hybridized amplified nucleic acid molecule by at most 2° C.

82. The kit of claim 80, further comprising at least one container comprising at least one nucleic acid molecule to be detected as a positive sample.

83. The kit of claim 80, further comprising a container comprising streptavidin bound to beads.
